(19) United States
(12) Patent Application Publication
(10) Pub. No.: US 2009/0163735 A1
(43) Pub. Date: Jun. 25, 2009

(54) APPARATUS AND PROCESS FOR CONTINUOUSLY PREPARING ETHYLENE CYANOHYDRIN

(75) Inventors: Volker Schleep, Einshausen (DE); Benedikt Laux, Monzernheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: EVONIK ROEHM GMBH, DARMSTADT (DE)

(21) Appl. No.: 12/296,780

(22) PCT Filed: Mar. 14, 2007
(86) PCT No.: PCT/EP2007/052402

§ 371 Date: Oct. 10, 2008

(30) Foreign Application Priority Data
Jun. 14, 2006 (DE) 10 2006 028 036.9

(51) Int. Cl. C07C 255/12; C07C 253/16; B01D 3/14

(52) U.S. Cl. 558/347; 558/451; 202/158

(57) ABSTRACT

The invention relates to an apparatus and to a process for continuously preparing ethylene cyanohydrin.

The invention relates to an apparatus and to a process for continuously preparing ethylene cyanohydrin.

The preparation of ethylene cyanohydrin from ethylene oxide and hydrocyanic acid is known. There are various solutions in the prior art for implementing this process on the industrial scale.

U.S. Pat. No. 2,653,162 describes the preparation of alkyl cyanohydrin by reacting alkyl oxides with hydrocyanic acid in the presence of a cation exchange resin with alkali metal carboxylate groups as the functional group. In a subsequent process step, the cation exchange resin has to be regenerated with a high level of cost and inconvenience.

U.S. Pat. No. 2,364,422 discloses a process for preparing alkyl nitrites by reacting tertiary alkyl epoxides with hydrocyanic acid. Cyanohydrin is formed in an intermediate stage and is hydrated immediately.

In DE1232570, ethylene oxide and liquid hydrocyanic acid are converted to ethylene cyanohydrin in alkaline medium by pumped circulation in a closed tube circuit and transferring the reaction mixture into a postreactor. In this process, turbulent conditions, a pH of above 8.5, a pressure of 1 to 10 at (gauge) and a temperature differing by not more than 5° C. over the entire reaction zone, which is in the range of 20-100° C., are employed.

It was an object of the invention to develop a process which enables virtually complete conversion of the reactants used in a continuous process, with simultaneously high space-time yield.

It was a further object of the invention to provide a suitable apparatus for performing the process.

The object is achieved by a process for continuously preparing ethylene cyanohydrin from ethylene oxide and hydrocyanic acid, characterized in that the reactants are fed in aqueous solution continuously to a reactor, the reaction is effected by means of a tube coil at a pressure of 11-25 bar and a temperature of 101-110° C., and the pH is controlled by means of the addition of sodium hydroxide solution.

It has been found that the process according to the invention can achieve conversions of 90-98%.

It has likewise been found that simple process control can be effected via the addition of sodium hydroxide solution. The unconverted reactants are stripped out. The resulting distillate is neutralized and then fed back to the reactor without further workup steps. The pH required for the reaction is established by the metering of sodium hydroxide solution into the reactor.

The reaction is effected under basic conditions. Preference is given to a pH between 8 and 13. This pH is established with bases, preferably with dilute sodium hydroxide solution. 1-50% sodium hydroxide solution, preferably 5-8% NaOH, is used.

The process is performed under elevated pressure, preferably at a pressure of 11 to 25 bar, more preferably at 12-14 bar. The temperature is controlled according to the prevailing pressure. The temperature range is between 100 and 110° C. Preference is given to working at a pressure of 12-14 bar and a temperature around 107° C. (107° C.±1° C.).

The distillate removed from the reaction mixture comprises hydrocyanic acid (0.1-30%), ethylene oxide (0.1-60%) and water (90-30%). The distillate is first neutralized. This is done with conventional acids. Preference is given to using formic acid. The pH of the distillate is adjusted to 3-4 with the acid and fed back to the reactor via the reactant stream.

The invention additionally relates to an apparatus for continuously preparing ethylene cyanohydrin, characterized in that, in a closed reactor system, the reactants are fed to a reactor whose working temperature is between 101 and 110° C., unconverted reactants are drawn off via a downstreams column, collected in a distillate vessel and then metered back into the reactor, and the crude product is drawn off at the column bottom. The product thus obtained is fed to further distillation stages for purification.

The unconverted reactants, for example hydrocyanic acid, ethylene oxide and water, are removed from the system via the top of the column. The top of the column has a temperature between 85-95° C. and a pressure of 900 to 1000 mbar.

At the bottom of the column, the crude product is removed at a temperature of 105-120° C. The crude product is purified in downstream distillation stages which free the crude product of low and high boilers.

As a result, the ethylene cyanohydrin is present in a very high purity. There are thus various possible uses. The ethylene cyanohydrin is preferably used as a precursor in the pharmaceuticals and cosmetics industry.

1. Process for continuously preparing ethylene cyanohydrin from ethylene oxide and hydrocyanic acid, characterized in that the reactants are fed in aqueous solution continuously to a reactor, the reaction is effected by means of a reactor column at a pressure of 11-25 bar and a temperature of 101-110° C., and the pH is controlled by means of the addition of sodium hydroxide solution.

2. Process according to claim 1, characterized in that the unconverted reactants are removed from the process by means of stripping and are fed directly back to the process.

3. Process according to claim 1, characterized in that a pressure of 12-14 bar is employed.

4. Process according to claim 1, characterized in that a temperature of 107° C. is employed.

5. Apparatus for continuously preparing ethylene cyanohydrin according to claim 1, characterized in that, in a closed reactor system, the reactants are fed to a reactor whose working temperature is between 101 and 110° C., unconverted reactants are drawn off via the top of the column, collected in a distillate vessel and then metered back into the reactor, and the crude product is drawn off at the column bottom and fed to further distillation stages for purification.

6. Apparatus for continuously preparing ethylene cyanohydrin according to claim 5, characterized in that the temperature in the top of the column is between 85-95° C. at 900 to 1000 mbar.

7. Apparatus for continuously preparing ethylene cyanohydrin according to claim 5, characterized in that the crude product is freed of low and high boilers in downstream further distillation stages.

8. Use of ethylene cyanohydrin prepared by the process according to claim 1 as a precursor in the pharmaceuticals and cosmetics industry.
